(19) United States
(12) Patent Application Publication
(10) Pub. No.: US 2011/0245926 A1
(43) Pub. Date: Oct. 6, 2011

(54) INTERVERTEBRAL SPACER AND METHODS OF USE

(76) Inventor: Michael S. Kitchen, Charleston, SC (US)

(21) Appl. No.: 13/074,488

(22) Filed: Mar. 29, 2011

Related U.S. Application Data
(60) Provisional application No. 61/319,543, filed on Mar. 31, 2010.

(51) Int. Cl. A61F 2/44

(52) U.S. Cl. 623/17.16

(57) ABSTRACT

An intervertebral implant device is presented. When placed between vertebral bodies of the mammalian spine, the device can effect fusion between the vertebral bodies. The implant is formed in layers in an arcuate geometry, wherein placement is facilitated by the implant first adopting a generally linear geometry, and through the process of placement reassumes its intrinsic arcuate form. The central axis of the spacer/implant may be oriented in a generally vertical direction, and lying generally parallel to the spinal axis. The implant may comprise shape memory materials to cause shape transition and formation of the device in situ between vertebral bodies.

Applicant claims the benefit of Provisional Application Ser. No. 61/319,543 filed Mar. 31, 2010.

### FIELD OF THE INVENTION

This invention relates to spinal stabilization generally, and is more particularly directed to devices or implants for surgical placement in the mammalian spine.

### SUMMARY OF THE INVENTION

An intervertebral implant device is presented. The implant is formed in layers in an arcuate geometry, wherein placement is facilitated by the implant first adopting a generally linear geometry, and through the process of placement reassumes its intrinsic arcuate form. The central axis of the spacer/implant may be oriented in a generally vertical direction, and lying generally parallel to the spinal axis. The implant may comprise shape memory materials to cause shape transition and formation of the device in situ between vertebral bodies.

### BACKGROUND OF THE INVENTION

Degenerative spine disease affects millions of Americans. Latest statistics suggest that in excess of 600 thousand surgical spine fusion procedures are performed in the U.S. annually. The clinical symptoms of degenerative spine disease directly causes millions of lost days at work and impacts the daily living of millions of young, middle-aged and elderly Americans impacting the U.S. population significantly in terms of financial consequence as well as in less tangible quality of life. Fusion procedures with instrumentation (implants) and artificial or native bone graft material are clinically proven to provide patients with measurably improved outcomes when compared to fusion procedures without implants using native bone graft alone. Patients experience greater pain control, faster return to work, and increased capacity to perform activities of daily living.

Minimally Invasive Surgery (MIS) has contributed substantially to surgical fields across a broad spectrum; providing better outcomes, expanding eligible patient populations, lessening peri-operative pain, shortening recovery times and allowing for unprecedented access to anatomy that conventional techniques will not permit. MIS has been widely adapted in the fields of General Surgery, G.I. Medicine, Cardiovascular Surgery, Neurovascular Surgery, Urology, and Gynecology to name a few. In general Orthopedic Surgery and Orthopedic Spine Surgery in particular have not experienced the same advances in MIS techniques for implant placement procedures. Largely this has been due to the unavailability of implants that are capable placement through small access devices and capable of providing the structural capacity required to meet stress requirements placed upon bones and joints. The orthopedic industry has been fully cognizant of the need for and benefits to be realized through adaptation of less invasive technologies.

Current intervertebral spacer implants are typically of the “VBR” or Vertebral Body Replacement type configuration. These devices are probably better referred to as intervertebral spacers which function as wedges or blocks placed between vertebral bodies. Functionally these implants serve to provide a rigid structure that when placed between vertebral bodies induces a healing process that results in the formation of a continuous boney connection between the vertebral endplates. Physiologically, rigid stabilization that permits very little motion to occur between adjacent vertebral bodies is probably crucial to the induction of bone formation across the intervertebral space. Interestingly, the natural course of degenerative spine disease eventually results in fusion between vertebral bodies. Surgical fusion accelerates the process largely by eliminating or very substantially reducing motion between spinal segments. Recent advances in the biology of fusion adjuncts including inductive proteins and artificial bone substrates have contributed significantly to the speed and reliability with which fusion will occur.

Bone growth promoting adjuncts to surgery are in use. The advent and practice of placing artificial bone graft material or various bone growth stimulating factors with spacers has increased fusion reliability to rates approaching those associated with cylindrical cages. Further the spacer devices can be placed through access smaller than that required for a cylindrical cage port.

VBR implants are characteristically comprised of materials that closely approximate bone density, geometry is typically of rectangular cross-section, and these implants are usually placed by wedging or hammering into the intervertebral space. Surgical access for VBR type devices is sized slightly larger than the smallest cross-section dimension of the implant. Much current emphasis has been placed on attempting to minimize cross-sectional area of the implant and reducing the size of required surgical access. As implants have become smaller there is always a concern that subsidence becomes a greater possibility, current designs probably approach the physical limits wherein failure through subsidence will become increasingly common.

The most frequently practiced surgical approach in the lumbar spine for placement for current designs is the Trans-Foraminal approach referred to as a TLIF procedure (Trans-foraminal Lumbar Interbody Fusion). This anatomic path offers several advantages for the patient and surgeon: the approach is posterior, the neuro-foramin is routinely dissected and nerve root decompressed as a part of this surgery, the spinal cord is typically avoided, and the patient does not require repositioning to place pedicle screws and rods. The surgical objectives of this procedure are multiple: often the disc space is accessed and material removed (discectomy), the nerve root is decompressed, an implant is placed into the intervertebral space, and posterior fixation (pedicle screws and rods) is achieved.

There are limitations to available implants and placement techniques. More particularly,
There is a need for an intervertebral spacer implant that can be placed with even smaller surgical access requirements.
There is a need for an intervertebral spacer implant that has a larger bearing surface area, minimizing the potential to subside into the vertebral endplates.
There is a need for an intervertebral spacer implant that can be placed utilizing the disc space access procedures that are generally familiar to spine surgeons.
There is a need for an intervertebral spacer implant that can correct anatomic deformities commonly associated with degenerative spine disease processes.
There is a need for an intervertebral spacer implant that is adaptable to placement utilizing a variety of anatomic approaches.
There is a need for dynamic stabilization implants that can be placed utilizing the least invasive manner.
There is a need for dynamic stabilization implants with little or no risk of being extruded from the disc space.

### DESCRIPTION OF THE DRAWINGS

FIG. 1 shows an anatomic approach for placement and demonstrates the relationship between major anatomic structures that impact the surgical approach.

FIG. 2 is anteriolateral view of deployment of the device in the spine, demonstrating the anatomic relationship of structures affecting deployment.

FIG. 3 shows the device being placed in situ within the intervertebral space.

FIG. 4 is an isolation of the device, showing the device as expanded to demonstrate the interlocking relationship of components.

FIG. 5 is view of a formed helical arrangement of the device showing areas of structural attenuation that allow the device to bend and form a helical shape without exceeding the strain capacity of the material.

FIG. 6 is a top, plan view of showing the device being deployed and forming in situ within the intervertebral space.

FIG. 7 shows the device in both a linear and annular forms.

FIG. 8 shows an embodiment of the device with an elastic sheath positioned over a shape memory core configuration.

FIG. 9 is enlarged isolation of an embodiment of the device wherein segments 206 are positioned on a shape memory core 212.

FIG. 10 is an embodiment to the device wherein a material forms an attenuated implant comprising wedge shaped segments positioned over a central core that is sized to provide shape memory function.

FIG. 11 is a plan view in isolation of a portion the device showing the wedge shaped segments, or “Pie Pieces,” over a shape memory core

FIG. 12 is an oblique view demonstrating placement of the device into the intervertebral space.

FIG. 13 is an embodiment of the device with folding shape memory components, with wedge shaped segments or “Pie Pieces” linked with bendable elements 213 between them and shown in a coiled position.

FIG. 14 shows the folding shape memory components of FIG. 13, with the device in a straight configuration and with folded portions extended.

FIG. 15 is an enlarged isolation of an embodiment of the device having shape memory components with an “I” shaped cross-section, and ribbon like structure.

FIG. 16 demonstrates an exemplary embodiment of a cannula, showing a cross-section that corresponds to a cross-section of the wedge shaped segments (“Pie Piece” shaped components), and additionally providing lumens for gas or fluid transport.

FIG. 17 shows an embodiment of the device formed as a straight configuration and demonstrating closure of outside spaces between wedge shaped segments or “Pie Piece” components.

FIG. 18 shows tapered and notched ends of an embodiment of the device, showing overlap at the ends that provide geometry for flat or dome shaped ends where vertebral endplates are contacted by the device.

FIG. 19 demonstrates a cross-section of an embodiment of a deployment catheter/cannula with implant positioned inside the catheter/cannula, and providing a cross-section profile that inhibits twisting of the implant during placement.

FIG. 20 demonstrates an embodiment of the invention within a central lumen of an exemplary deployment cannula, which is shown in straight linear geometry corresponding to low temperature state of a shape memory material such as NiTinol.

FIG. 21 depicts an exemplary deployment catheter/cannula providing a resistance heating element and central implant channel.

FIG. 22 shows the deployment catheter/cannula of FIG. 21 with an embodiment of the device exiting from the tip of the catheter/cannula, with transition occurring at a zone defined by resistance heating elements.

FIG. 23 shows a lateral side and top of a deployment cannula embodiment, with cut away portions providing clearance for extruding the device.

FIG. 24 is a view of a multiple segment implant comprising a plurality of shape memory material components that link the segments.

FIG. 25 is an enlarged view of the shape memory material linking components of FIG. 24, demonstrating mid-portions thereof structurally attenuated in a single plane.

FIG. 26 is an embodiment of the device shown in linear geometry with shape memory material links between segments, with each link shown as transitioned to a flexed shape.

FIG. 27 shows individual shape memory material linking components in a flexed shape on the right of the figure and a straight shape on the left of the figure.

FIG. 28 is an embodiment comprised of a continuous shape memory material component, with selected areas structurally attenuated and shown in a flexed configuration.

### DESCRIPTION OF THE PREFERRED EMBODIMENTS

In preferred embodiments, the present invention is an implant or spacer that may be placed in a mammalian spine. The implant may be used for assisting fusion of the spine. The implant or spacer comprises an arcuate member having at least two layers. The layers are positioned one over the other in essentially a stacked arrangement. The arcuate member may be helical, as shown in FIG. 4, to produce the layers. In other embodiments, the layers are stacked one over the other, preferably in an interlocking or interdigitating manner, as shown in the drawings. Each layer of the arcuate member is substantially annular. The term “annular” as used herein includes both the helical configuration shown in FIG. 4, and configurations where one layer is stacked over an adjoining layer. The term “annular” includes layers wherein there is a small gap between a first end of the arcuate layer and an opposite end of the arcuate layer. In most embodiments, the first layer and second layer, and additional layers if used, are concentric. In most embodiments, the central axis of the arcuate member, and the central axis of the layers, is positioned generally parallel to the spinal axis.

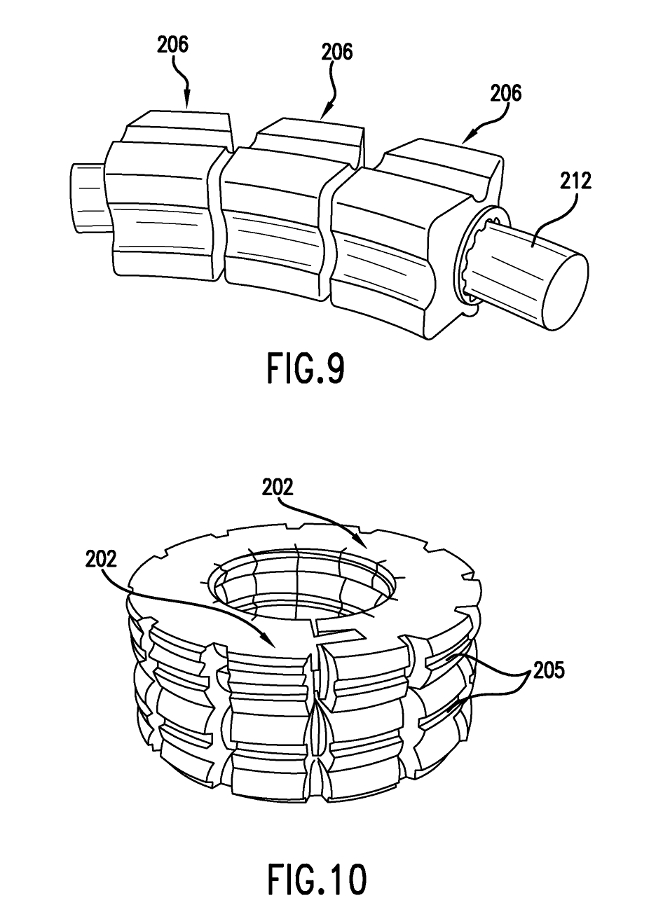

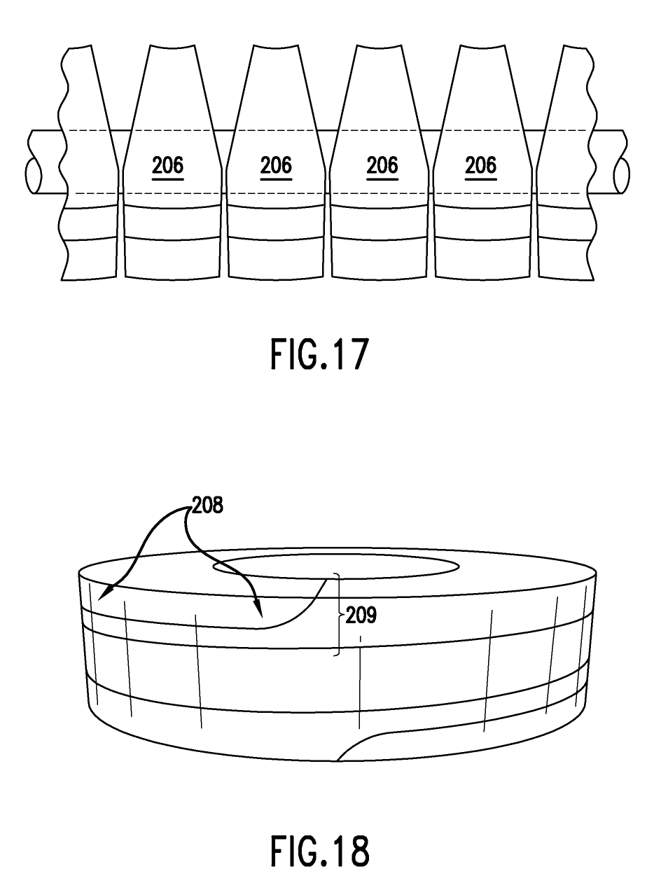

In some embodiments as presented herein, the implant is a continuous elongated and arcuate member. In other embodiments, the implant is formed by a plurality of wedge shaped members that are connected or linked by a linking element, or in other embodiments, a plurality of linking elements. The wedge-shaped, or “Pie Piece” shaped, segments or members allow the arcuate implant or spacer to be formed as a straight for placement, and assume an overall arcuate, or annular, shape upon placement in the spine. The “wedge” or “Pie Piece” shape includes shapes that are like those of FIGS. 9, 11, 13, 14 and 17, wherein, in plan view, a generally larger one end tapers to a generally smaller oppose end. In each of these embodiments, when viewed in plan, the wedge-shape does not have a point formed by acute angle, but rather, the end is truncated. In embodiments where the layers are formed by wedge shaped members, each layer should comprise at least four (4) wedge-shaped members, and it is preferred that each layer comprise at least eight (8) wedge-shaped members.

In a preferred embodiment, the invention employs shape memory material(s) to effect construction of an intervertebral spacer in situ between vertebral bodies of the mammalian spine. The implant may be placed utilizing a novel deployment method referred to as the Thermal Method of Deployment described in U. S. Pat. No. 7,582,109 issued Sep. 1, 2009, which effects an ordered, sequential and predictable introduction of heat to a thermally active shape memory material in linear form, causing the shape memory material to transition to a predetermined size and shape as the implant is spatially transitioned through a controlled zone of temperature differential. Placement may be effected through a Minimally Invasive Surgical technique (MIS) wherein the device is initially formed in a collapsed indeterminate linear form.

The shape memory component of preferred embodiments is initially formed as straight, and may be essentially a wire. A temperature differential of heat (or cold) is introduced at the tip of the deployment catheter and the device self deploys to a determinate higher (or lower) temperature complex geometric shape having super elastic (or rigid) properties.

Alternatively, the implant may comprise shape memory material having super-elastic characteristics at temperatures at or below human body temperature, and structural properties that permit deformation of the implant to a linear form utilizing mechanical force alone. This embodiment relies upon the shape memory properties of the implant to recover shape set form as the implant passes through, and emerges from the tip of, a deployment catheter or cannula.

Collectively, the embodiments of the device presented herein address the need for an intervertebral spacer device that can be placed into the disc (intervertebral) space. Commonly practiced surgical access procedures utilized in spine surgery may be employed for positioning the device. The implant may be positioned in the spine through access achieved in performing a discectomy procedure that utilizes a posterior approach. Without being bound by theory, placement of the device does not require additional removal of tissue or bone to achieve access sufficient for deployment.

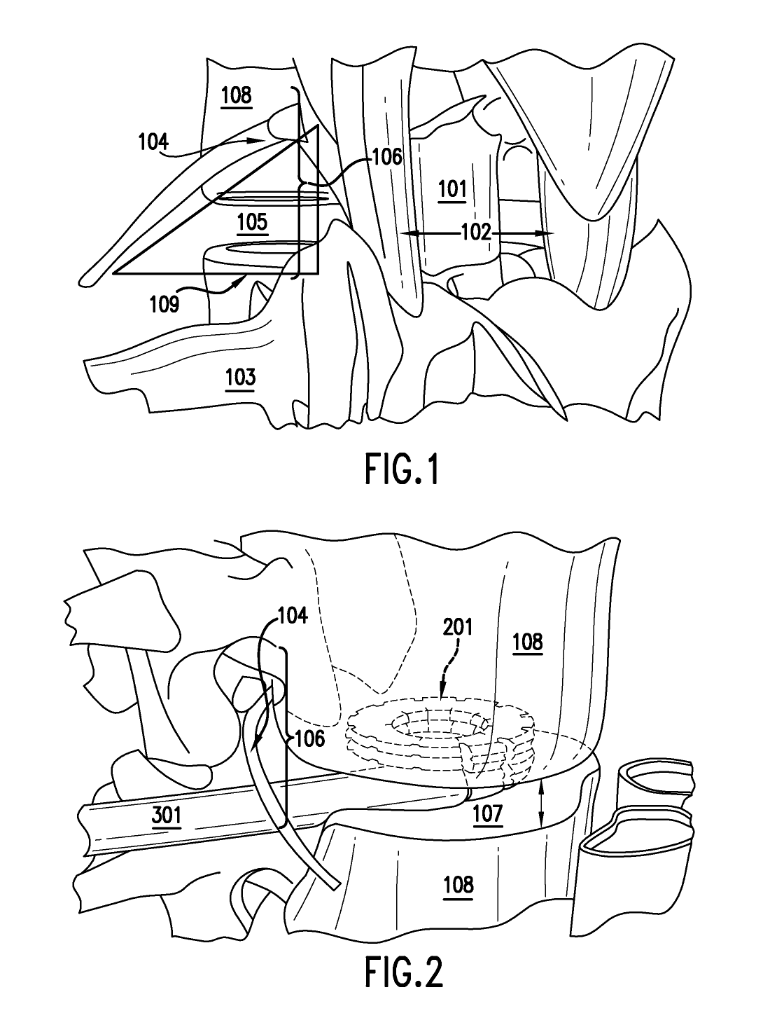

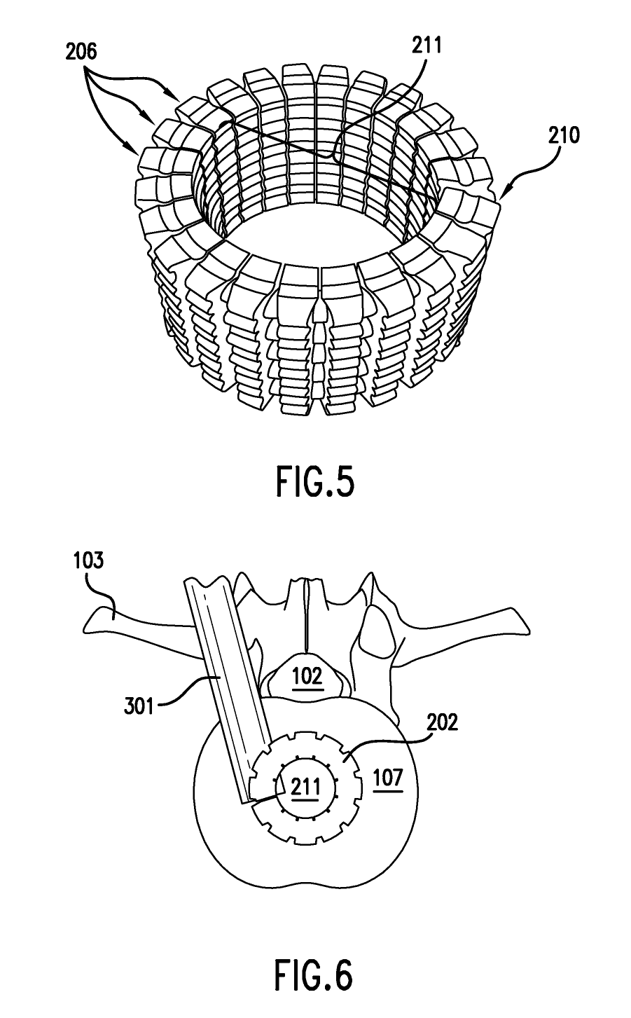

The preferred embodiments of the invention are positioned between adjacent vertebral bodies. FIGS. 1, 2, 3, 6 and 12. Using currently known surgical techniques, the surgeon prepares the intervertebral space or disc space 105 in a conventional manner, performing a discectomy and vertebral endplate preparation. Access for this procedure may be achieved through the neuroforamina 106 with the disc space 105 entered just caudal to the segmental nerve root 104. In fusion procedures, this approach is referred to as a TLIF or Trans-Foraminal Lumbar Interbody Fusion procedure. Typical access utilizing this anatomic approach yields a roughly triangular opening 109 to the disc space which measures 10 mm to 12 mm horizontally and 8 mm to 10 mm vertically. FIG. 1. This space is defined laterally by the spinal canal 102 and neuroforamina 106, inferiorly by the transverse process 103 of the immediately caudal vertebrae, and superiorly by the segmental nerve root 104. These anatomic boundaries yield a natural limit for deployment cannula size and shape at the time of placement. If an implant or required deployment device is larger than the anatomically defined space a variable amount of bone will have to be removed to gain access, or a more extensive dissection of soft tissue is performed in order to retract the nerve root out of the access pathway.

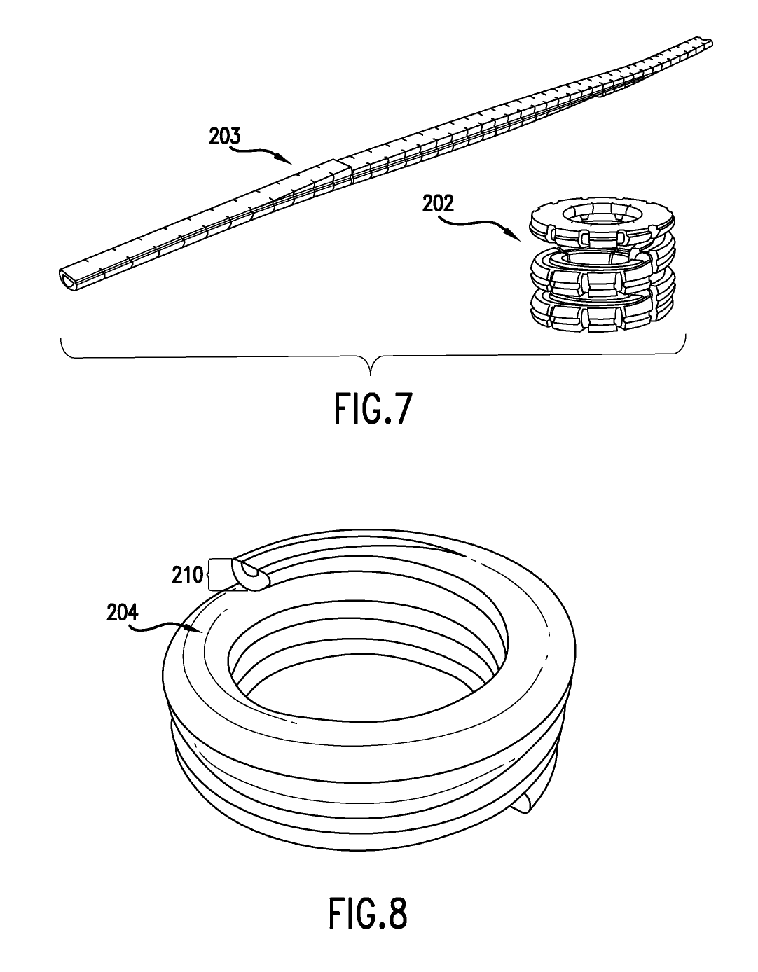

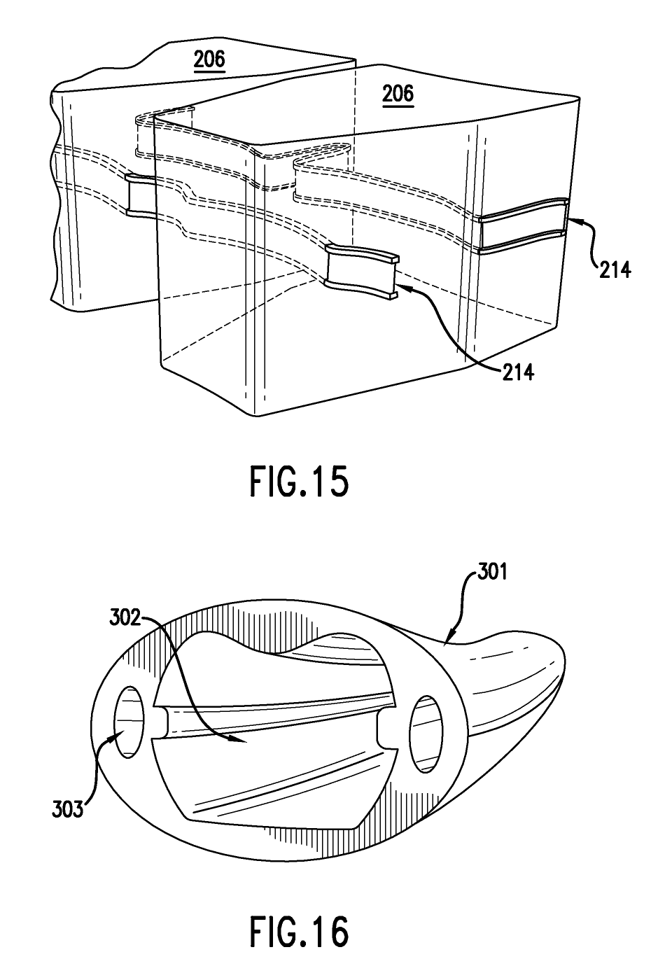

In a preferred embodiment, the implant may be deformed to adopt a linear form at low temperatures and prior to placement. FIGS. 7, 17, 20 and 26. While in a linear form, due to sufficiently low temperatures or due to subjecting the device to a straightening force (stress induced transition), the implant may be placed into the central lumen of a deployment catheter or cannula 302. The catheter or cannula is preferred to have at least one lumen with a cross-sectional profile that matches the cross-section of the implant. FIG. 15. Tolerances are designed in this cross-section of the central lumen 302 to allow for passage of the linear formed implant 203 along the elongated or longitudinal axis of the catheter or cannula, while additionally providing space for flow or transport of gases or liquids, such as chilled liquid coolant. In one embodiment, liquid coolant is circulated in a continuous manner through a central lumen 302 such that it surrounds the implant and maintaining a low temperature, thus inhibiting the implant from heating prematurely and transitioning to its higher temperature shape. The catheter or cannula may also have a secondary lumen 303 or lumens intended to allow flow of gases or liquids such as coolants in a counter circulatory direction, thereby permitting evacuation of accumulated coolant from the placement site.

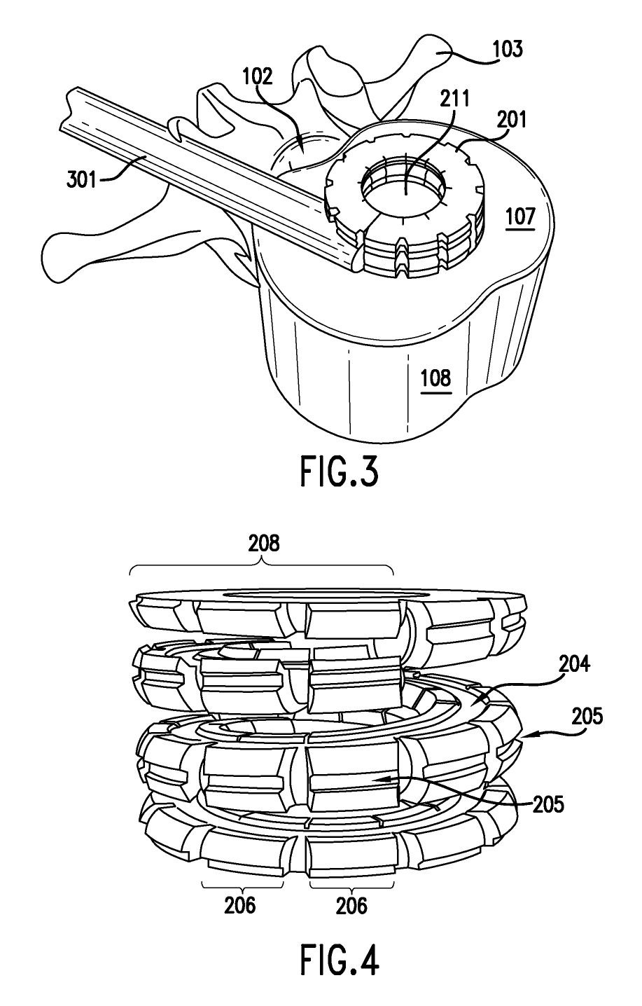

A technology that will permit controlled transition of shape memory material implants at the tip of the catheter or cannula is described in U.S. Pat. No. 7,582,109 entitled “Thermal Transition Methods and Devices,” issued Sep. 1, 2009. Shape memory materials, and in particular, shape memory alloys, and notably NiTinol alloys, have the property of differing atomic structures which are intrinsic to temperature (energy) state. For NiTinol alloys, low energy states (Martensite state) are characterized by having properties of malleability and a non-superelastic form; at these low temperature (energy) states a device or implant has an indeterminate shape: such a device will adopt the geometry or shape it is deformed to. For purposes of proposed designs, low temperature states correspond with the capability to deform the implant to a linear geometry 203. This deformation may be accomplished with manual force. Therefore, a surgeon can manually bend or deform a helical implant into a roughly linear form that may be placed into the central lumen of the deployment catheter or cannula, and subsequently advance the device along the central lumen of the catheter or cannula by manual force. FIGS. 3, 6 and 12.

In some embodiments, the implant comprising shape memory material may be forced into a deployment catheter or cannula utilizing manual force to overcome the super-elastic resistance of the implant. In such an embodiment, the implant will return to shape set geometry as it emerges from the tip of the deployment catheter or cannula within the intervertebral space. This process exploits the stress induced transition property of shape memory materials.

Contrary to the shape indeterminate low temperature state of shape memory materials, in the higher temperature state, the alloy exerts force towards achieving a predetermined final shape, or “set shape” (FIG. 10.) that was imparted during manufacture. For NiTinol alloys, the high temperature state is defined as “Austenite”. In the Austenite state, the material has super-elastic properties and is capable of exerting considerable force directed towards achieving its shape set form upon reaching its required, preset transition temperature. The specific temperature at which NiTinol alloys transition between Martensite and Austenite states may be controlled through manufacturing processes. It is possible, by appropriate design and production of the device, to set a temperature for the transition of shape to occur at or below body temperatures, such that the implanted device will remain in a super-elastic state as long as the subject mammal's body at the point of implantation is maintained at (or above) the body temperature for that species of mammal.

U.S. Pat. No. 7,582,109 describes the process of applying heat to a shape memory material device in a “controlled, ordered and sequential” manner at a spatially defined zone within a catheter, cannula 305 or similar apparatus to produce transition between temperature states of said shape memory implant or device. This technology provides the capability to move an implant through a catheter or similar device placed to achieve access in a minimally invasive manner with transition of the shape memory implant occurring at the tip or a defined portion of the deployment device (catheter). FIGS. 2, 3, 6 and 12. For the present invention, this process may provide a controlled temperature milieu (relatively cool) proximal to the specified point of transition by immersing the implant in a continuously circulated chilled fluid (saline solution). Both the implant and the circulated fluid are heated at the defined point 304 of transition to a temperature above the transition temperature specific to the shape memory implant. In a preferred embodiment this temperature transition occurs at the tip of the deployment cannula. Heat is transferred to the circulated cold saline solution and the implant in a sequential manner as the coolant flows from the tip of the cannula, and the implant is manually pushed forward through the cannula. Heat may be produced utilizing an electrical resistance heating element incorporated into the tip of the cannula 304, effecting a localized and defined zone of temperature differential 305. The zone of temperature differential is preferred to occur within a spatially defined area, or more precisely, in a curved three dimensional surface of definable thickness. This process causes the implant to adopt its pre-determined shape set size and shape 202 in situ, forming the realization of the implant. FIGS. 3, 6, 12, and 22. Excess fluid coolant is removed from the implantation site through secondary channels 303 provided for in the cannula design.

In a preferred embodiment, the implant comprises a shape memory alloy, and more preferably, NiTinol. Certain NiTinol alloys possess temperature dependent shape memory characteristics. At a relatively low specific temperature, when the alloy is in the Martensite state, it is malleable and of indeterminate shape 203. When heated to a higher temperature, the implant transitions to the Austenite state, and adopts a determinate shape 202, and exhibiting super-elastic properties. The temperatures at which these transitions occur are defined as follows: for Martensite states, a specific temperature at which all of the metal is in the Martensite state is defined as Mf—Martensite final; for relatively higher Austenite states the temperature at which all of the implant transitions to the superelastic Austenite state is defined as Af—Austenite final. Specific Mf and Af temperatures can be determined by design and production of the alloy at the time of manufacture. These temperatures can be changed by varying alloy composition and heat treating processes. Typically, Mf and Af temperatures are separated by 10° to 25° Celsius. This differential defines the so called “Hysteresis Curve,” a double sigmoidal curve, and the area between the curves correlates to energies of activation. Af temperatures can be specified within a range of ±3° to 5° Celsius, and can be specified at ranges that are less than human body temperature. For the preferred embodiment, Af temperatures will be specified to maintain superelasticity to 5° to 10° Celsius below normal body temperatures.

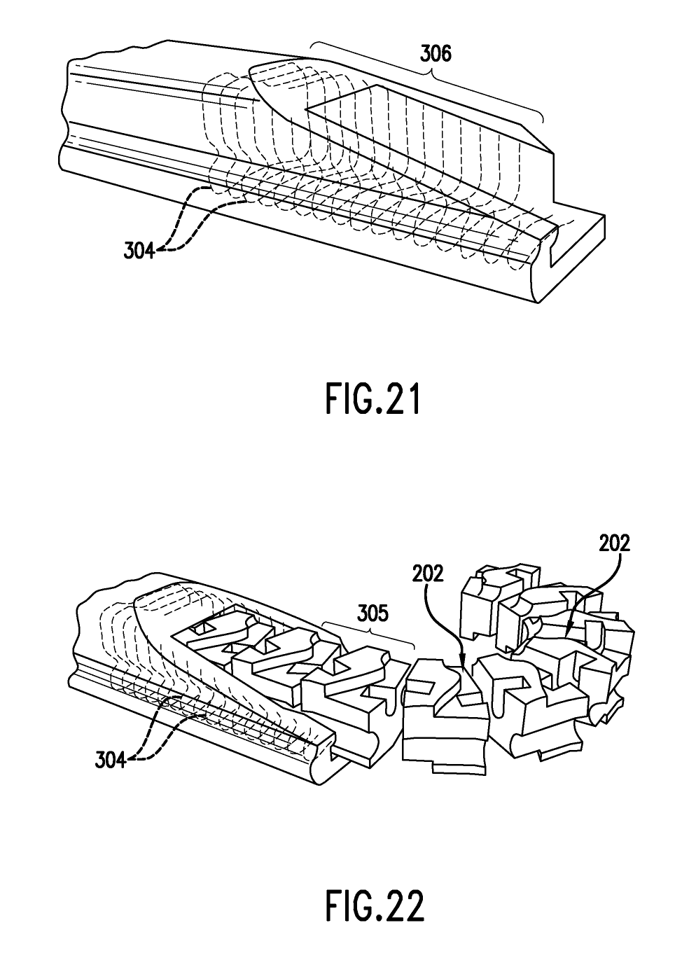

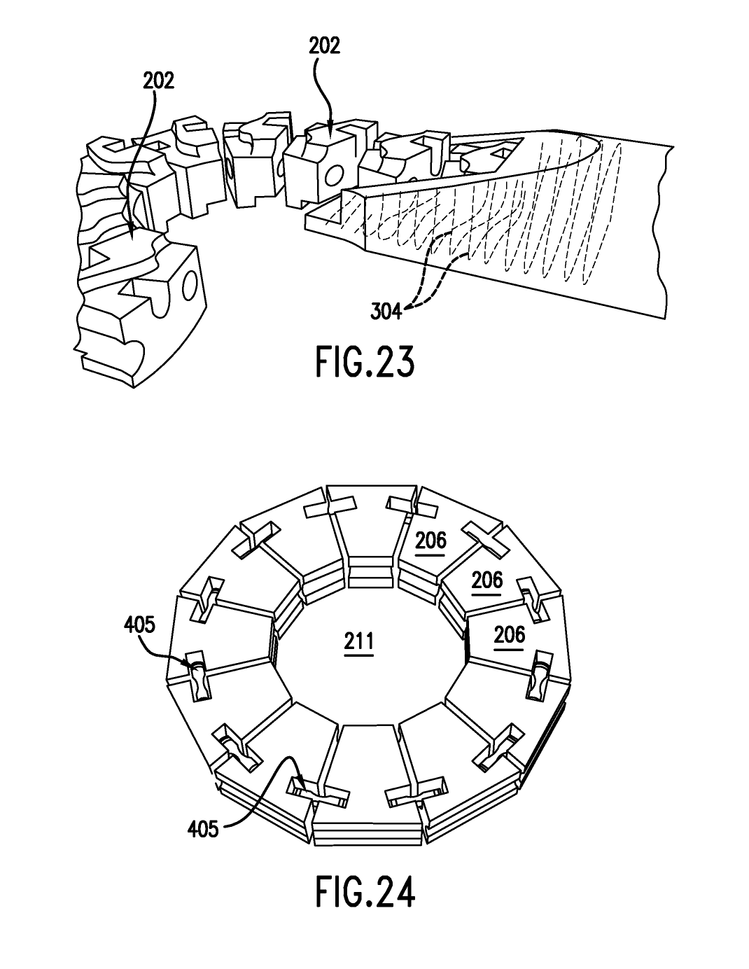

In a preferred embodiment, the device is a single homogeneous element comprising shape memory alloy. FIG. 10. This single element adopts a single helical geometry when deployed between vertebral bodies with the axis of the helix parallel to the spinal axis. FIGS. 1, 2, 3, 6 and 12. Structurally, the implant is attenuated in a radial manner having wedge shaped or “pie piece shaped” segments that reduce strain upon the device when it is straightened for placement, such as through the deployment catheter or cannula. FIGS. 2, 3, 6, 12, 16, 20, 21, 22 and 23. The cuts or spaces between “pie piece shaped” segments permit angular deformation between the segments as the device transitions between linear geometry and annular, coiled and/or helical geometry, thereby reducing mechanical strain upon the material. The cross-section of this embodiment of the device is configured so that the upper portion and the corresponding lower portion of the coils interlock in a male/female relationship—an interdigitating “tongue and groove” profile 204. This arrangement provides a structural link between individual segments of the coils, and prevents sliding between the coils in shear. Additionally, this relationship facilitates centering of one coil above the next as the device exits the deployment catheter or cannula for positioning in the spine. Once placed, the implant produces an essentially rigid structure in compression, extending between the endplates of the vertebral bodies. The interdigitating relationship is shown in FIG. 4, where the device is shown in a vertically expanded state.

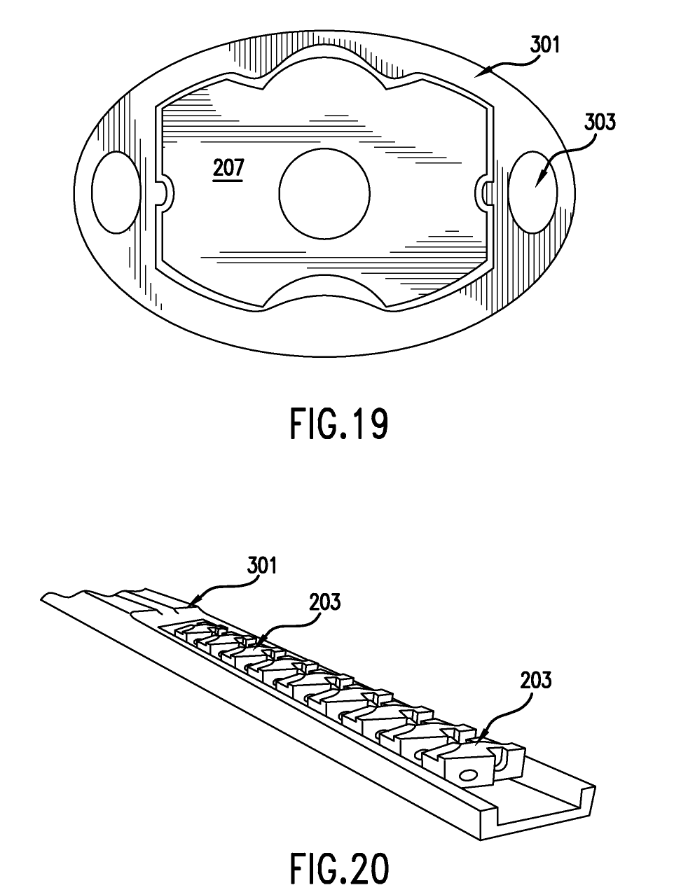

Ends of the implant may be tapered 208 and/or notched 209 along the longitudinal axis to produce flat or domed shaped end portions of the implant where contact to bone is made. FIGS. 5, 16 and 18. These geometric features are produced in some embodiments by using a helical geometry with variable pitch or slope. FIG. 18. Implants without tapering and notching are shown for comparison. FIG. 5, 8. Without these features, a gap or step off 210 is present at the ends of the implant, which does not produce a matched interface with the vertebral endplates. The side profiles of the individual segments are preferred to be notched as well. This geometric feature allows the device to pass through a matched deployment catheter or cannula and interlock with the cross-section preventing the implant form twisting relative to the deployment cannula at deployment, and allowing the surgeon to manipulate it axially. FIGS. 16 and 19.

In another embodiment, the implant is comprised of composite construction, wherein a shape memory helical core (FIGS. 9, 11, and 17) is surrounded by polymer segments. The shape memory core of this embodiment provides the motive force for deployment, and the polymer segments provide structural elements resistant to compressive forces parallel to the spinal axis. This embodiment provides the capability to select materials having capabilities to promote bone growth and match material properties of bone or disc.

A composite structure of this design allows less complex processes of manufacture. The polymer components may be molded, and the shape memory material component may be constructed with a less complex cross-section than with a single all shape memory material design. FIGS. 7 and 10. Individual segment pieces for this configuration may be vertically stacked and aligned, or alternate segments may be designed so that there is a staggered relationship vertically, where one segment at a higher layer overlaps and bears loads across two or more segments at a lower level layer.

Another embodiment comprises a wire shape memory material core within a polymer component. FIG. 8. This embodiment may have a solid NiTinol core surrounded by a covering, which preferably has elastic properties, and which may be a tube or coating comprising rubber or plastic that is placed over an elongated memory material element, such as wire, and such as NiTinol wire. As shown, the superior and inferior surfaces of the “rubber tube” have profiles that match, allowing the surfaces to interlock 204. These interlocking profiles of superior and inferior surfaces of the implant provide for lateral stability that resists shear forces.

If less elasticity is desired, the implant may be constructed with a segmented geometry having radially placed vertical cuts or gaps formed through the elastic component. This design yields a structure wherein the polymer segments are wedge shaped, and fit together as “pieces of a pie”. FIGS. 9 and 10.

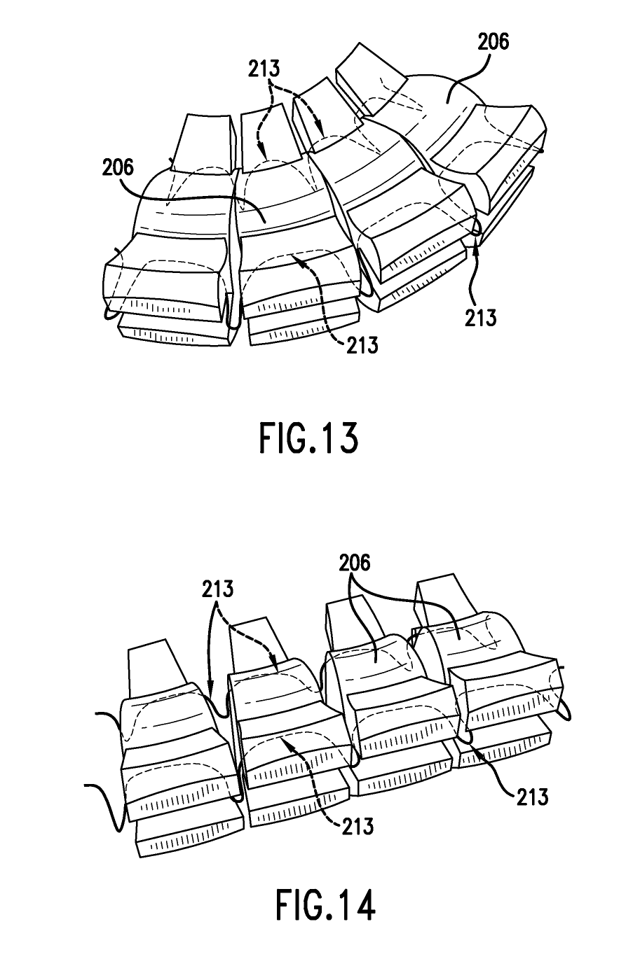

In one embodiment, the device comprises a plurality of wedge or pie piece shaped components 206 that are linked with one or more linking elements 213 positioned between them. The linking elements may be wire, and may be of light gage and bendable. The overall construct is straightened for passage through the lumen of the deployment device, with the linking elements forcing the overall arcuate shape of the device upon exiting the deployment device. FIGS. 13 and 14. In an embodiment, the linking elements impart spring biasing. The use of spring biasing means that temperature dependent transitioning of shape memory material is not required. Temperature dependent transitioning of shape memory material may be used in another embodiment.

The linking elements or linking structure(s) of the embodiments that employ linking elements may be a continuous structural section in the form of an “I” or “T” section 214. FIG. 15. In another embodiment, the linking elements or linking structures are wire, and may have a round cross section 213. FIGS. 13 and 14. Folds or bends in the linking structure of an embodiment as shown are formed at the time of manufacture. Shape memory is imparted to the overall shape of the device by the linking structure, so that the shape corresponds fully to the desired wound helical form of the implant as it is placed in the spine. The small structural elements may be deformed using manual force for placement of the device into a deployment catheter or cannula of straight or curved geometry. An attachment may be made at the proximal end of the implant at the time of placement to a rigid or semi-rigid linear instrument (deployment control rod), which is released upon placement of the implant. The linking structures may be formed of shape memory material, which may be NiTinol.

The portion of the linking element that is embedded into the segment is insulated and protected from changing shape with temperature or stress. Without being bound by theory, it is believed that this structure form a more secure and stable connection. A continuous linear shape memory material component linking more than two segments may be more likely to fail when the shape memory component transitions shape, since, at a very local level, the bond may be more likely to fail.

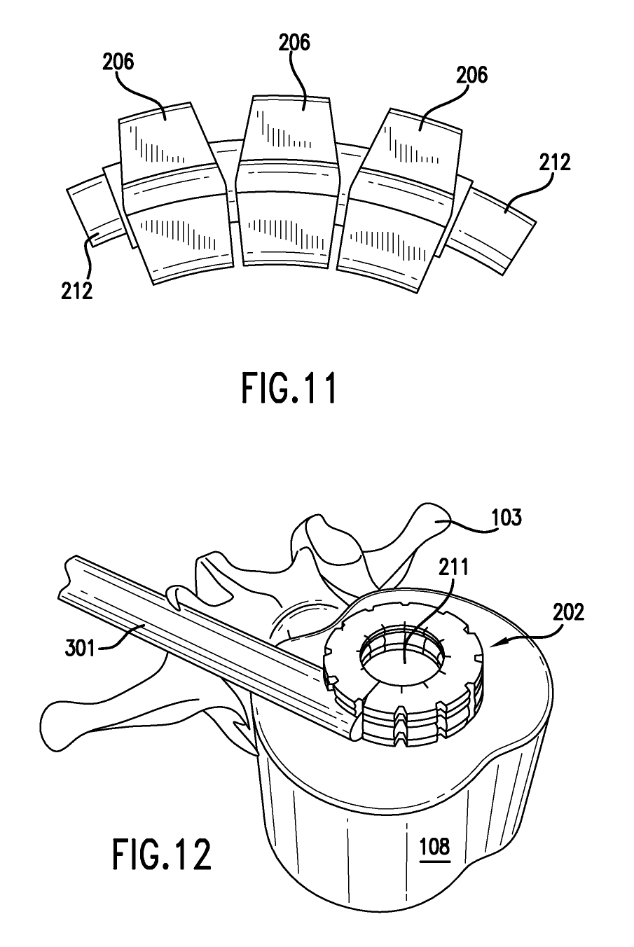

An embodiment may be manufactured utilizing a homogeneous shape memory alloy composition, wherein the entire implant is machined or cast as a single piece (FIG. 4, 10) or the wedge or pie shaped elements are manufactured separately, and then joined with the foldable shape memory elements, yielding an implant constructed of a single material. This configuration allows for the wedge or “pie” shaped components 206 to be composed of materials that differ from the shape memory components 212. FIGS. 11 and 13. This compositional configuration permits the use of materials having desirable characteristics that may extend beyond currently available shape memory materials. Such capabilities may include but are not limited to: materials with modulus of elasticity similar to bone, materials with elastic characteristics allowing for deformation under physiologic loads, materials having biologic properties capable of stimulating bone proliferation, materials that are radio-lucent, materials derived from human or animal tissues, materials that are artificial bone substitutes, and/or materials having anti-microbial properties.

In another embodiment, the device has a discontinuous design that may incorporate shape memory materials. In this embodiment, the shape memory component supplies the motive force effecting transition between linear and formed geometries of the implant. The shape memory material may be comprised of a plurality of similar or dissimilar elements. In this embodiment the implant transitions between an essentially linear geometry 203 and a helical geometry 202. The implant is placed through a tubular deployment cannula in a substantially linear form that leads to the intervertebral space. The device transitions to a shape set helical form at the tip of the deployment catheter (FIG. 22, 23) within the intervertebral space. Motive force of transition may be provided through use of thermal properties of shape memory materials, or stress induced transition at constant temperature.

Configurations of designs employing a plurality of shape memory components provide certain advantages in comparison to designs that employ singular shape memory components or multiple shape memory components that extend through multiple segments within the implant. “Continuous” shape memory elements—those that extend through or across two or more segments of the implant—require connections that are designed to allow movement of the shape memory element relative to the segment and may limit the strength or type of connection that can be made. As a specific example: for an implant with helical multilayered geometry with a continuous helical “wire” shape memory material element (FIG. 8) the shape memory material element will transition between linear geometry and curved helical geometry in a continuous manner. This property leads to shape change in the element in a continuous manner along the length of the shape memory element; essentially all parts must move in relation to the segmental components as transition occurs. This shape change makes forming a direct mechanical connection between the shape memory material element and a polymer composition segment difficult as every portion of the shape memory material element will change its shape through the transition process.

In one embodiment, limited points or areas are provided where transition in shape occurs, leaving intervening segments where material shape change does not occur through transition, and where mechanical connections can be made. A shape memory element, which may be a NiTinol element, having varied physical properties along its length (FIG. 28) may be employed. In this embodiment, the polymer segments of the implant may be molded or otherwise positioned over the shape memory material component, such that contact occurs at those portions of the shape memory material component that are not subjected to transition as the implant moves between linear 203 and coiled forms. This embodiment comprises selective structural attenuation 401 at portions of the shape memory material. This feature provides controlled bending at specified points. The shape memory material component may be subjected to one or more of several possible shape configuration or surface treatment techniques to enhance bonding strength between the polymer segment and the shape memory material component. Shape designs may include but are not limited to: “T” members, “H” members, “mushroom”, or “barbell” type ends 403 configured into the non-transitioning portions of the shape memory material component. The shape is desired to provide enhanced anchoring strength. The surface of the non-transitioning portions of the shape memory material components may also be textured 402 or roughened to provide enhanced bonding between the polymer segment and shape memory material components.

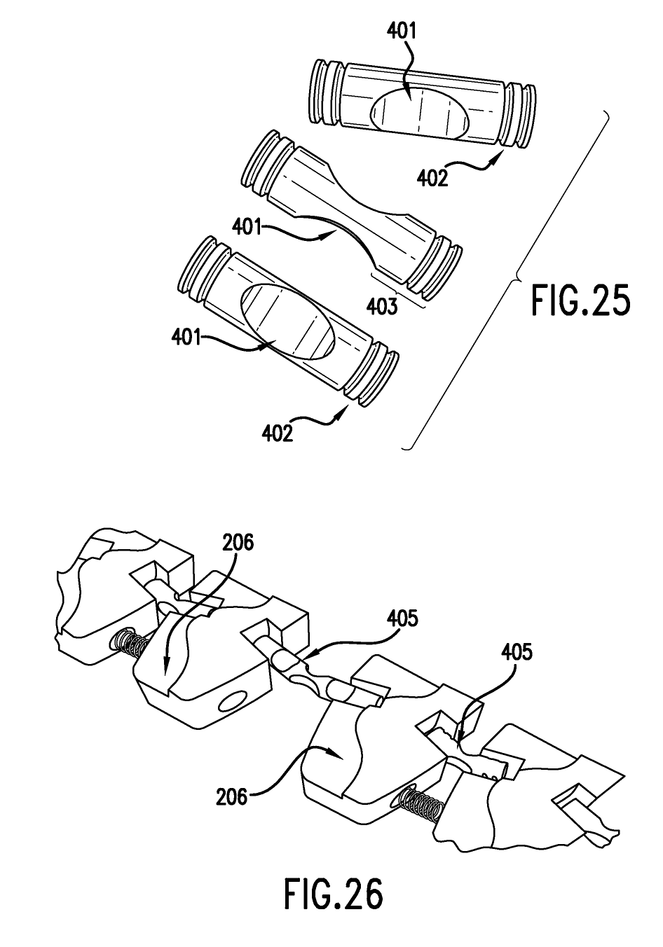

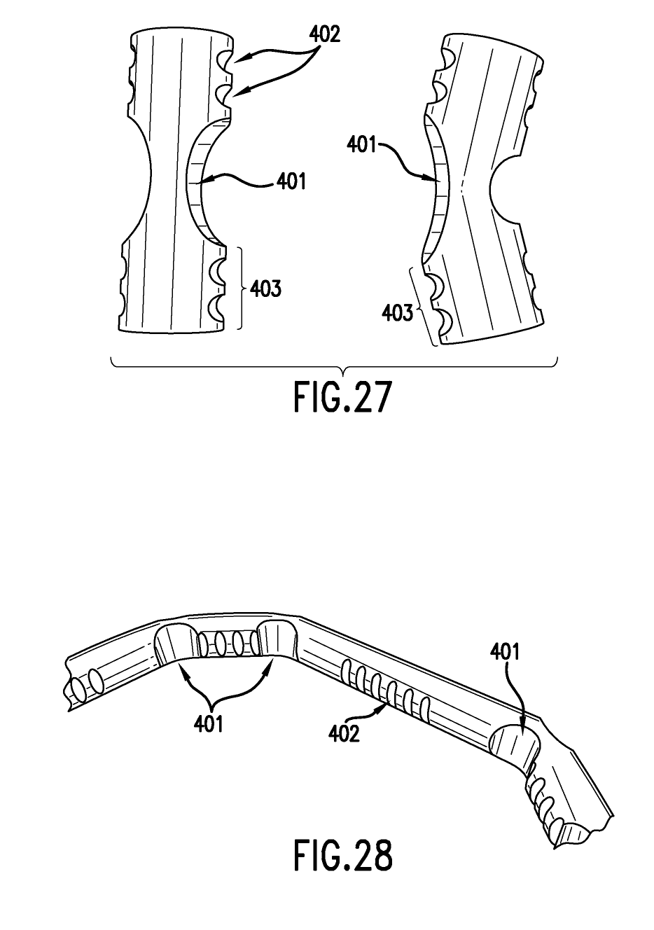

A further embodiment comprises separate shape memory components that form the physical links between each polymer segment of the implant. FIG. 24. This embodiment allows shape memory material components to be manufactured in large numbers with a high degree of precision and measured physical properties through a process described hereinafter. Each shape memory material component 405 is preferred to allow deformation of the individual components in a proscribed manner. FIGS. 25 and 27. Components may be designed to allow for favored deformation in specific planes of motion. FIG. 25. This configuration allows the individual components to be deformable either through temperature dependent means, or through the process of stress induced transition at specific spatially defined portions of the component 401. Typically, the component may be configured in a “barbell” shape 403 with the middle portion of the component deformable 401, and the ends configured to be significantly less deformable under strain than the middle portion, which deforms to provide the arcuate shape of the device. This embodiment provides for secure and reliable high strength bonding at the interface between the polymer segment and the shape memory material components at the ends of the component where deformation does not occur. Bonding may be achieved by molding the polymer segment over the shape memory material components or welding or gluing processes.

In another embodiment, the linking elements that are present between segments 206 is a separate shape memory component providing for solid connections at portions contacting polymer segments (FIG. 24, 26) and for an intervening section having transition properties and mechanical strength characteristics permitting shape changes to accommodate transition of the overall implant between linear (FIG. 26) and coiled (FIG. 24) geometries. This plurality of shape memory components allows for ease of manufacture, since the shape memory material components (FIG. 25, 27) may be manufactured in large quantities with a high degree of mechanical and dimensional precision. Manufacturing may be specified to produce large numbers of these components having a precisely controlled degree of deformation that occurs in response to application of a specific force at a specific temperature. The manufacturing process may involve serial removal of material from the middle portion of the shape memory material while intermittently subjecting the component to a specified force, and measuring deflection as the component is machined. This process may be accomplished utilizing liquid coolant for the machining process and laser or EDM cutting techniques. With low mass components, any heat added to the system in the machining process will be washed out with liquid coolant in a fraction of a second allowing for extremely rapid machining and measurement feedback cycles as the shape memory component is produced. This technique of machine manufacturing describes a technique wherein parts are manufactured to meet a specific mechanical performance parameter rather than a dimensional specification.

Each of the embodiments will induce rigid boney fusion to occur between the instrumented vertebral bodies when placed for this purpose. The invention may also be configured to allow for a degree of movement between vertebral bodies if desired, for example, if used for dynamic stabilization of the spine.

What is claimed:

1. A spinal implant for placement in a mammalian spine, comprising an arcuate member comprising a first arcuate layer and a second arcuate layer that is present over the first arcuate layer, wherein the arcuate member is constructed and arranged to be formed as generally straight for placement and transportation through a lumen, and wherein the arcuate member is constructed and arranged to assume an arcuate shape comprising the first arcuate layer and the second arcuate layer upon exiting the lumen.

2. A spinal implant for placement in a mammalian spine according to claim 1, wherein the first arcuate layer and the second arcuate layer are annular.

3. A spinal implant for placement in a mammalian spine according to claim 1, wherein the first arcuate layer and the second arcuate layer are annular and concentric.

4. A spinal implant for placement in a mammalian spine according to claim 1, a central axis of the arcuate member is oriented in a generally vertical direction, and the central axis is generally parallel to the spinal axis

5. A spinal implant for placement in a mammalian spine according to claim 1, wherein the arcuate member comprises:
a plurality of wedge shape members, and
a linking element that links the plurality of wedge shaped members,
wherein the linking element forms the arcuate member to the arcuate shape comprising the first arcuate layer and the second arcuate layer upon exiting the lumen.

6. A spinal implant for placement in a mammalian spine according to claim 1, wherein the arcuate member comprises:
a plurality of wedge shape members, and
a plurality of linking elements, wherein a linking element of the plurality of linking elements is present between two adjoining wedge shaped members of the plurality of wedge shaped members, thereby joining the plurality of wedge shaped members together,
wherein the linking elements form the arcuate member to the arcuate shape comprising the first arcuate layer and the second arcuate layer upon exiting the lumen.

7. A spinal implant for placement in a mammalian spine according to claim 1, wherein the arcuate member is helical.

8. A spinal implant for placement in a mammalian spine according to claim 1, wherein the arcuate member comprises a core comprising shape memory properties.

9. A spinal implant for placement in a mammalian spine according to claim 1, wherein the arcuate member comprises a core comprising thermal shape memory properties, and wherein the core forms the arcuate member to the arcuate shape comprising the first arcuate layer and the second arcuate layer upon exiting the lumen in response to a temperature change from a first temperature of the core when the core is in one portion of the lumen to a second temperature when the core exits the lumen.

10. A spinal implant for placement in a mammalian spine according to claim 1, wherein the arcuate member comprises:
a plurality of wedge shape members, and
a linking element comprising thermal shape memory properties that links the plurality of wedge shaped members,
wherein the linking element forms the arcuate member to the arcuate shape comprising the first arcuate layer and the second arcuate layer for positioning the arcuate member in the spine, and wherein the linking element forms the arcuate member to the arcuate shape comprising the first arcuate layer and the second arcuate layer in response to a temperature change from a first temperature of the linking element to a second temperature of the linking element.

11. A spinal implant for placement in a mammalian spine according to claim 1, wherein the arcuate member comprises:
a plurality of wedge shape members, and
a plurality of linking elements comprising thermal shape memory properties, wherein a linking element of the plurality of linking elements is present between two adjoining wedge shaped members of the plurality of wedge shaped members, thereby joining the plurality of wedge shaped members together,
wherein the linking elements form the arcuate member to the arcuate shape comprising the first arcuate layer and the second arcuate layer for positioning the arcuate member in the spine, and wherein the linking elements form the arcuate member to the arcuate shape comprising the first arcuate layer and the second arcuate layer in response to a temperature change from a first temperature of the linking elements to a second temperature of the linking elements.

12. A spinal implant for placement in a mammalian spine according to claim 1, wherein the arcuate member comprises:
a plurality of wedge shape members, and
a linking element that links the plurality of wedge shaped members,
wherein the linking element forms the arcuate member to the arcuate shape comprising the first arcuate layer and the second arcuate layer upon exiting the lumen by spring biasing.

13. A spinal implant for placement in a mammalian spine according to claim 1, wherein the arcuate member comprises:
a plurality of wedge shape members, and
a plurality of linking elements, wherein a linking element of the plurality of linking elements is present between two adjoining wedge shaped members of the plurality of wedge shaped members, thereby joining the plurality of wedge shaped members together,
wherein the linking elements form the arcuate member to the arcuate shape comprising the first arcuate layer and the second arcuate layer upon exiting the lumen by spring biasing.

14. A spinal implant for placement in a mammalian spine according to claim 5, wherein the first arcuate layer comprises four (4) wedge shaped members.

15. A spinal implant for placement in a mammalian spine according to claim 6, wherein the first arcuate layer comprises four (4) wedge shaped members.

16. A spinal implant for placement in a mammalian spine according to claim 1, wherein the first arcuate layer and the second arcuate layer comprise a slope relative to a central axis of the arcuate member.

17. A spinal implant for placement in a mammalian spine according to claim 1, wherein the arcuate member comprises:
a plurality of wedge shape members, and
a linking element comprising thermal shape memory properties that links the plurality of wedge shaped members,
wherein the linking element forms the arcuate member to the arcuate shape comprising the first arcuate layer and the second arcuate layer for positioning the arcuate member in the spine, and wherein the linking element forms the arcuate member to the arcuate shape comprising the first arcuate layer and the second arcuate layer in response to a temperature change from a first temperature of the linking element to a second temperature of the linking element, wherein the temperature change occurs in a defined temperature zone.

18. A spinal implant for placement in a mammalian spine according to claim 1, wherein the arcuate member comprises:
a plurality of wedge shape members, and
a plurality of linking elements comprising thermal shape memory properties,
wherein a linking element of the plurality of linking elements is present between two adjoining wedge shaped members of the plurality of wedge shaped members, thereby joining the plurality of wedge shaped members together,
wherein the linking elements form the arcuate member to the arcuate shape comprising the first arcuate layer and the second arcuate layer for positioning the arcuate member in the spine, and wherein the linking elements form the arcuate member to the arcuate shape comprising the first arcuate layer and the second arcuate layer in response to a temperature change from a first temperature of the linking elements to a second temperature of the linking elements, wherein the temperature change occurs in a defined temperature zone.
